(19) United States
(12) Patent Application Publication
(10) Pub. No.: US 2023/0355423 A1
(43) Pub. Date: Nov. 9, 2023

(54) SECUREMENT SYSTEMS AND METHODS FOR FLUID COLLECTION ASSEMBLIES

(71) Applicant: PUREWICK CORPORATION, Covington, GA (US)

(72) Inventors: Florence Stevenson, Baltimore, MD (US); Michael Bielawa, Baltimore, MD (US); Brian Murphy, Baltimore, MD (US); Carolyn Rose, Covington, GA (US); Charlotte Lux, Covington, GA (US); Alexis Dorsey, Covington, GA (US); Alisa Rantanen, Covington, GA (US); Eric Alan Fallows, Covington, GA (US); Seth Schneider, Covington, GA (US); Alexis Nunn, Covington, GA (US); Hannah Kriscovish, Covington, GA (US)

(21) Appl. No.: 18/246,121

(22) PCT Filed: Sep. 22, 2021
(86) PCT No.: PCT/US2021/051456

§ 371 Date: Mar. 21, 2023

Related U.S. Application Data
(60) Provisional application No. 63/082,261, filed on Sep. 23, 2020.

(51) Int. Cl. A61F 5/44; A61F 5/455; A61F 5/453; A61F 5/443

(52) U.S. Cl. CPC A61F 5/4408; A61F 5/4556; A61F 5/453; A61F 5/4401; A61F 5/443

(57) ABSTRACT

Embodiments disclosed herein are fluid collection assemblies including one or more securement elements and methods for collecting fluids with the fluid collection assemblies. In an embodiment, a fluid collection assembly includes a fluid collection device and means for securing the fluid collection device to the user with a fluid permeable body of the fluid collection device positioned proximate to the urethra of the user through the opening in a fluid impermeable barrier of the fluid collection device.

### CROSS-REFERENCE TO RELATED APPLICATIONS

This application claims priority to U.S. Provisional Patent Application No. 63/082,261 filed on Sep. 23, 2020, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections. While external catheters improve user comfort, leaks may occur due to size of the user, movement of the external catheter, and/or improper placement of the external catheters.

Thus, users and manufacturers of fluid collection devices continue to seek new and improved devices, systems, and methods to collect urine.

### SUMMARY

Embodiments disclosed herein are fluid collection assemblies including one or more securement elements and methods for collecting fluids with the fluid collection assemblies. In an embodiment, a fluid collection assembly includes a fluid collection device defining a chamber, an opening having fluid communication with the chamber and configured to be placed proximate to a urethra of a user, and an aperture configured to receive a conduit therethrough to provide fluid communication between the chamber and the conduit. The fluid collection assembly also includes means for securing the fluid collection device to the user with the opening positioned proximate to the urethra of the user.

In an embodiment, a method of collecting fluid from a user includes providing a fluid collection device including a fluid impermeable barrier defining a chamber, an opening having fluid communication with the chamber and configured to be placed proximate to a urethra of a user, and an aperture configured to receive a conduit therethrough to provide fluid communication between the chamber and the conduit. The method also includes securing the fluid collection device to the user with the opening positioned proximate to the urethra of the user. The method also includes collecting fluid in the chamber of the fluid collection device.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device, according to an embodiment.

FIG. 1B is a front view of a female user wearing the fluid collection device of FIG. 1A.

FIG. 1C is an exploded isometric view of the female fluid collection device of FIG. 1A.

FIG. 1D is a cross-sectional view of the female fluid collection device of FIG. 1A taken along line 1-1 thereof, according to various embodiments.

FIG. 2A is an isometric view of a female user wearing the fluid collection device of FIG. 1A with a securement system, according to an embodiment.

FIGS. 2B and 2C are front views of a securement system for a fluid collection device, according to an embodiment.

FIG. 2D is an isometric view of a female user wearing the fluid collection device of FIG. 1A with a securement system, according to an embodiment.

FIG. 2E is an isometric view of a female user wearing the fluid collection device of FIG. 1A with a securement system, according to an embodiment.

FIG. 2F is an isometric view of a female user wearing the fluid collection device of FIG. 1A with a securement system, according to an embodiment.

FIG. 2G is an isometric view of a female user wearing the fluid collection device of FIG. 1A with a securement system, according to an embodiment.

FIG. 2H is an isometric view of a female user wearing the fluid collection device of FIG. 1A with a securement system, according to an embodiment.

FIG. 2I is a top view of a securement system for a fluid collection device, according to an embodiment.

FIGS. 2J and 2K are front views of a securement system for a fluid collection device, according to an embodiment.

FIG. 3A is an isometric view of a female user wearing the fluid collection device of FIG. 1A with a securement system, according to an embodiment.

FIG. 3B is an isometric view of a female user wearing the fluid collection device of FIG. 1A with a securement system, according to an embodiment.

FIG. 3C is an isometric view of a female user wearing the fluid collection device of FIG. 1A with a securement system, according to an embodiment.

FIG. 3D is an isometric view of a female user wearing the fluid collection device of FIG. 1A with a securement system, according to an embodiment.

FIG. 3E is an isometric view of a female user wearing the fluid collection device of FIG. 1A with a securement system, according to an embodiment.

FIG. 4A is an isometric view of a female user wearing the fluid collection device of FIG. 1A with a securement system, according to an embodiment.

FIG. 4B is an isometric view of a female user wearing the fluid collection device of FIG. 1A with a securement system, according to an embodiment.

FIG. 4C is an isometric view of a gasket of a securement system for use with a fluid collection device, according to an embodiment.

FIG. 4D is a side view of the gasket of FIG. 4C.

FIG. 5 is an isometric view of a female user wearing the fluid collection device of FIG. 1A with a securement system, according to an embodiment.

FIG. 6 is an isometric view of a fluid collection system, according to an embodiment.

FIG. 7 is a flow diagram of a method to collect fluid, according to an embodiment.

FIG. 8 is a block diagram of a system for fluid collection, according to an embodiment.

### DETAILED DESCRIPTION

Embodiments disclosed herein are securement systems for fluid collection devices, and methods of collecting fluid. Fluid collection devices such as external catheters improve user comfort over conventional internal catheters. Leaks, however, may occur with external catheters due to the size of the user, movement of the external catheter, and/or improper placement of the external catheters. Means and systems for securing the fluid collection device to the user disclosed herein are configured to provide the technical effect that results in positionally proper and convenient securement of a fluid collection device, such as an external catheter, in a desired position over the urethra of the user. This securement of the fluid collection device also provides the technical effect of preventing or inhibiting leaks and/or movement of the fluid collection device during use.

Embodiments of the fluid collection devices disclosed herein are configured to collect fluids from an individual. The fluids collected by the fluid collection devices may include urine. The fluids collected by the fluid collection devices may also include at least one of vaginal discharge, penile discharge, reproductive fluids, blood, sweat, or other bodily fluids. Embodiments of the securement systems and fluid collection devices disclosed herein may be used in fluid collection systems. The fluid collection systems may include a fluid collection device, a fluid storage container, and a portable vacuum source. Fluid (e.g., urine or other bodily fluids) collected in the fluid collection device may be removed from the fluid collection device via a conduit which protrudes into an interior region of the fluid collection device. For example, a first open end of the conduit may extend into the fluid collection device to a reservoir or chamber therein. The second open end of the conduit may extend into the fluid storage container or the portable vacuum source. The suction force may be introduced into the interior region of the fluid collection device via the first open end of the conduit responsive to a suction (e.g., vacuum) force applied at the second end of the conduit. The suction force may be applied to the second open end of the conduit by the portable vacuum source either directly or indirectly.

Fluid collection devices described herein may be shaped and sized to be positioned adjacent to the opening of a female urethra, positioned over a male penis, or otherwise used for fluid collection from male or females. Although the drawings illustrate embodiments generally used with fluid collection devices on a female, fluid collection devices configured for use with a male or female may be used with fluid collection assemblies described herein. In some embodiments, the fluid collection device may include a fluid impermeable barrier at least partially defining a chamber (e.g., interior region of the fluid collection device) of the fluid collection device. The fluid impermeable barrier also defines an opening extending therethrough from the external environment. The opening may be positioned adjacent to a female urethra or have a male urethra positioned therethrough. The fluid collection device may include a fluid permeable body disposed within the fluid impermeable barrier. The conduit may extend into the fluid collection device at a first end region, through one or more of the fluid impermeable barrier, fluid permeable body to a second end region of the fluid collection device. Exemplary fluid collection devices for use with the systems and methods herein are described in more detail below.

In some embodiments, the portable vacuum source may be disposed in or on the fluid collection device. In such embodiments, the conduit may extend from the fluid collection device and attach to the portable vacuum source at a first point therein. An additional conduit may attach to the portable vacuum source at a second point thereon and may extend out of the fluid collection device, and may attach to the fluid storage container. Accordingly, a vacuum (e.g., suction) may be drawn through the fluid collection device via the fluid storage container. Fluid, such as urine, may be drained from the fluid collection device using the portable vacuum source.

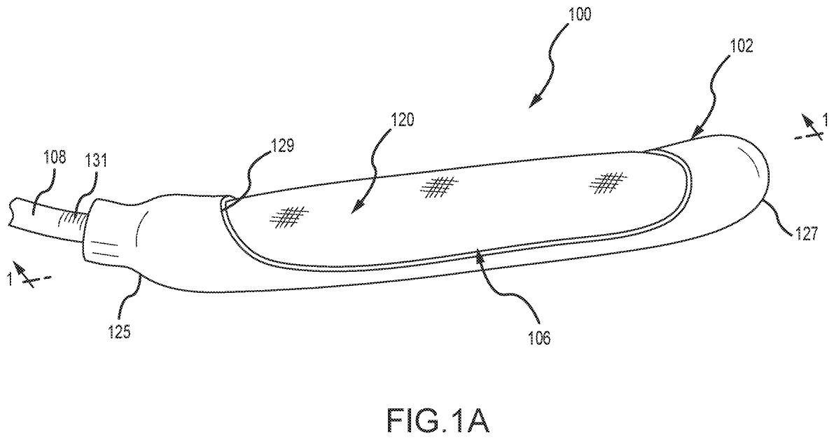

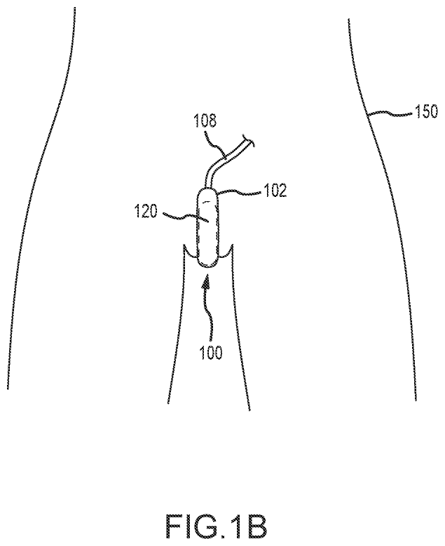

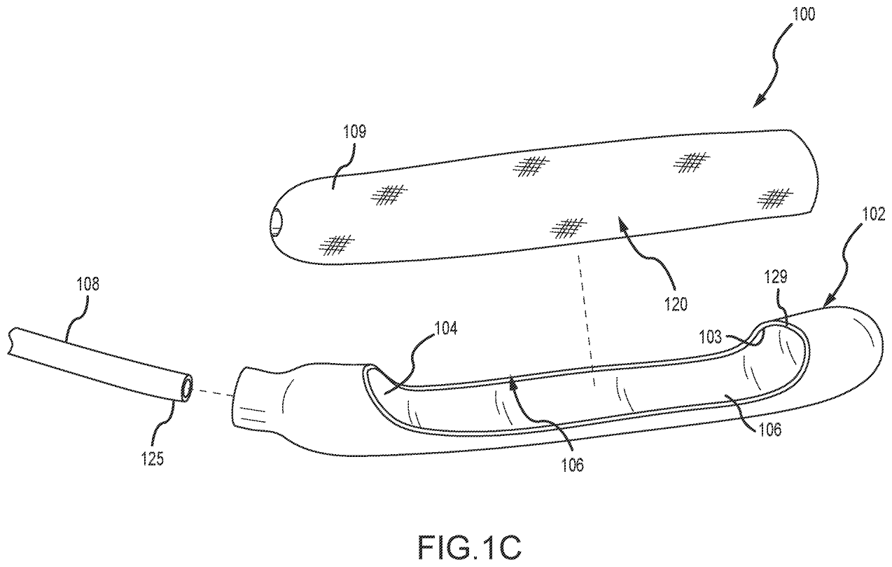

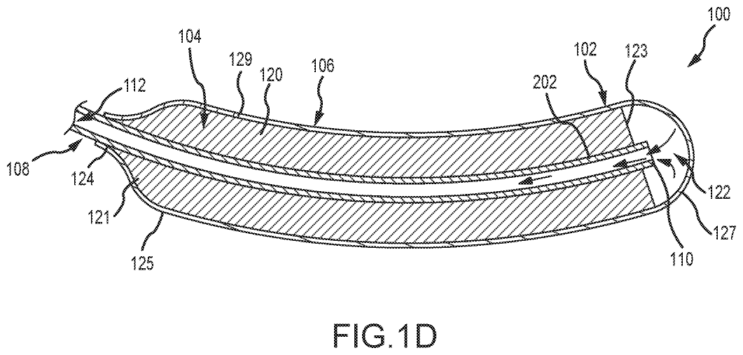

In some embodiments, one or more (e.g., all) of the securement systems disclosed herein may include or otherwise be configured to secure or position a fluid collection device on a user. FIGS. 1A-1D show a fluid collection device 100 that may be used with any of the securement systems disclosed herein, according to some embodiments. FIG. 1A is an isometric view of a fluid collection device 100, according to an embodiment. The fluid collection device 100 is an example of a female fluid collection device 100 that is configured to receive fluids from a female. The fluid collection device 100 includes a fluid impermeable barrier 102 having a first end region 125 and a second end region 127. The fluid impermeable barrier 102 at least partially defines a chamber 104 (e.g., interior region, shown in FIG. 1C) and includes an inward border or edge 129 defining an opening 106. The fluid impermeable barrier 102 is substantially cylindrical in shape between the first end region 125 and the second end region 127. In other embodiments, the fluid impermeable barrier 102 may include other shapes, such as one of more substantially planar surfaces, triangular, or other suitable shape. The opening 106 is formed in and extends longitudinally through the fluid impermeable barrier 102, thereby enabling fluids to enter the chamber 104 from outside of the fluid collection device 100. The opening 106 may be configured to be positioned at least proximate (e.g., adjacent to, interfacing, or contacting) the opening of a female urethra or over a buried penis of a male.

With the fluid collection device 100 may positioned at least proximate to the opening of the female urethra or over a buried penis, urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arc portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 can be configured to wick and/or allow transport of fluid away from the opening 106 towards a reservoir 122 and/or an inlet 110 of the conduit 108. The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” or other physical properties may exclude absorption into the fluid permeable body 120, such as not include absorption of the bodily fluid into the fluid permeable body 120. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 120 (e.g., absorbency), such as less than about 30 wt % of the dry weight of the fluid permeable body 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 210. In an embodiment, the fluid permeable body 210 may include at least one absorbent or adsorbent material.

The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir 122 (shown in FIG. 1D) in the chamber 104 and/or the inlet 110 of the conduit 108. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 1C) of the fluid permeable body 120 interfaces with an inner surface 103 (shown in FIG. 1C) of the fluid impermeable barrier 106. In some embodiments, at least a portion of the singular porous material of the fluid permeable body 120 extends continuously between the opening 106 and the reservoir 122 to wick any fluid from the opening 106 directly to the reservoir 122. Moreover, as the fluid impermeable barrier 102 is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106. In these and other embodiments, the fluid permeable body 120 includes an outer surface and a single layer or type of material between the opening 106 and the conduit 108 positioned within the fluid permeable body 120.

In some embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable body, with both the fluid permeable membrane and the fluid permeable body being disposed in the chamber 104. The fluid permeable membrane may cover or extend across at least a portion (e.g., all) of the opening 106. The fluid permeable membrane may be configured to wick any fluid away from the opening 106, thereby preventing the fluid from escaping the chamber 104. In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating.

The fluid permeable membrane may also wick the fluid generally towards an interior of the chamber 104. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous spun nylon fiber structure and a fluid permeable wicking membrane including gauze at least partially enclosing the spun nylon fiber structure. For example, the fluid permeable body 120 may include a gauze or other wicking fabric positioned to contact the skin of the user through the opening 106. In some embodiments, the gauze or other wicking fabric is wrapped around a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

FIG. 1B is a front view of a fluid collection device 100 in use on a female user 150. In use, the fluid permeable body 120 of the fluid collection device is positioned at least proximate to a urethra of the user 150. The fluid permeable body 120 is disposed within a chamber 104 (shown in FIG. 1D) of the fluid impermeable barrier 102 of the fluid collection device 100 and is exposed to the urethra of the user 150 through the opening 106 in the fluid collection device 100. The fluid collection device 100 may be secured to the user with any of a number of securement systems disclosed herein. Fluids received in the chamber 104 of the fluid collection device 100 from the urethra may be removed through the conduit 108.

FIG. 1D is a cross-sectional view of the fluid collection device 100 taken along line 1-1 of FIG. 1A. The fluid collection device 100 also includes the conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 extends past the second body end 123 and is positioned in the reservoir 122. However, in other embodiments, the inlet 110 may be positioned flush with or behind the second body end 123 of the fluid permeable body 120 that partially defines the reservoir 122. In some embodiments, the second body end 123 extends to the second end region 127 to substantially fill the chamber 104 and cover the inlet 110. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 may store fluids in the reservoir 122 therein. The reservoir 122 may be an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 1D, the reservoir 122 is defined by the second body end 123 of the fluid permeable body 120 and the second end region 127 of the fluid impermeable barrier 122. However, the reservoir 122 may be located at different locations in the chamber 104. For example, the reservoir 122 may be located at the end of the chamber 104 that is closest to the outlet 112. In these and other embodiments, the conduit 108 may extend through the first end region 125 of the fluid impermeable barrier 102 and to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108. In some embodiments, the fluid permeable body 120 fills or occupies substantially all of the chamber 104, including filling or occupying substantially all of the reservoir 122 is between the inlet 110 and the second end region 127 of the fluid impermeable barrier 102. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 1D. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). For example, an end (e.g., the inlet 110) of the conduit 108 may be generally flush or coplanar with the second body end 123 of the fluid permeable body 120. In other embodiments, the end of the conduit 108 may be recessed from the second body end 123 of the fluid permeable body 120. The end (e.g., the inlet 110) of the conduit 108 also may be selectively moveable between partially extending into the reservoir 122 (shown in FIG. 1D) and recessed from or flush with the second body end 123 of the fluid permeable body.

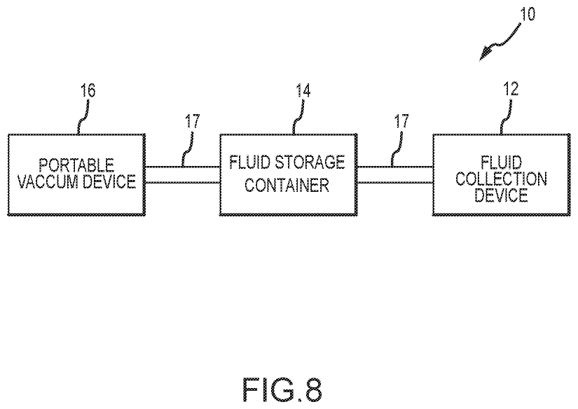

When secured to the fluid collection device 100, the conduit 108 is configured to provide fluid communication with and at least partially extend between one or more of a fluid storage containers and a portable vacuum source, as described below with FIG. 8. For example, the conduit 108 may be configured to be fluidly coupled to and at least partially extend between one or more of the fluid storage containers and the portable vacuum source. In an embodiment, the conduit 108 is configured to be directly connected to the portable vacuum source. In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container or the portable vacuum source. In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 1A) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a thermoplastic elastomer (TPE) polymer.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

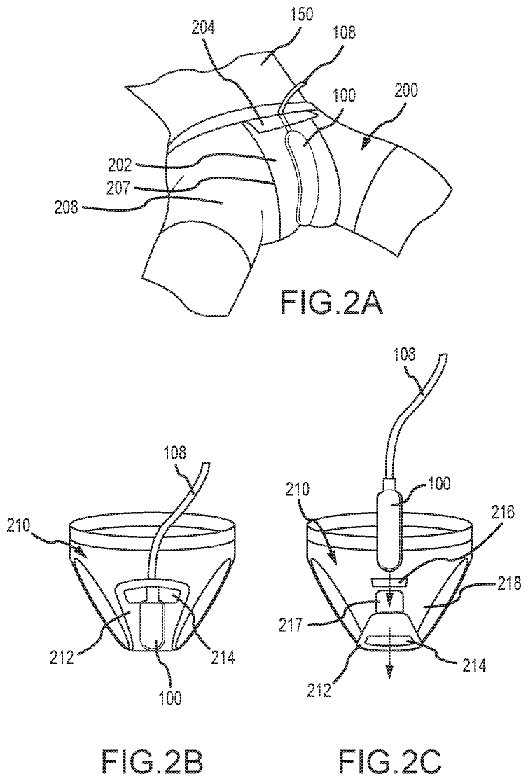

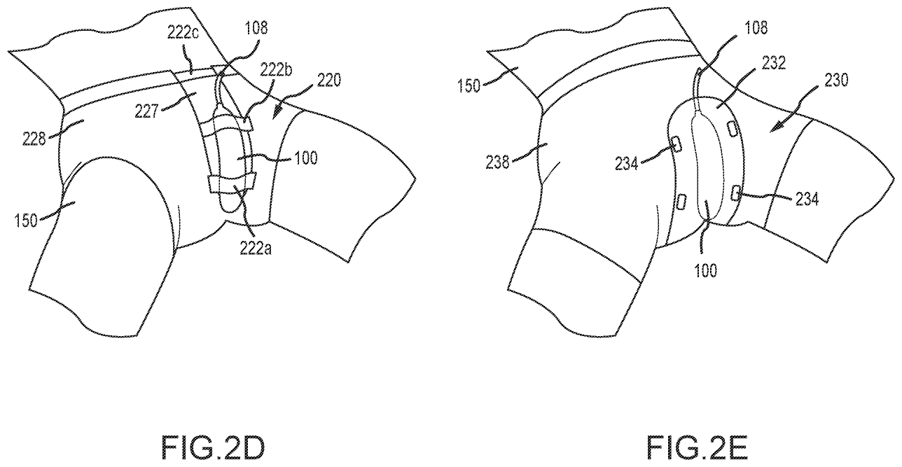

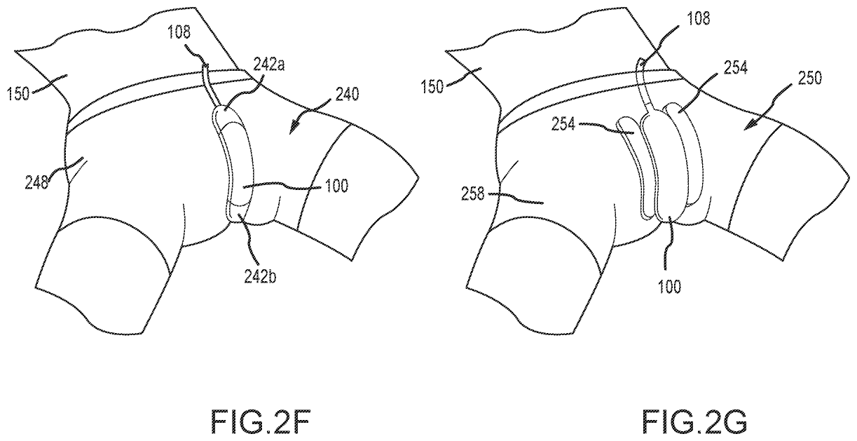

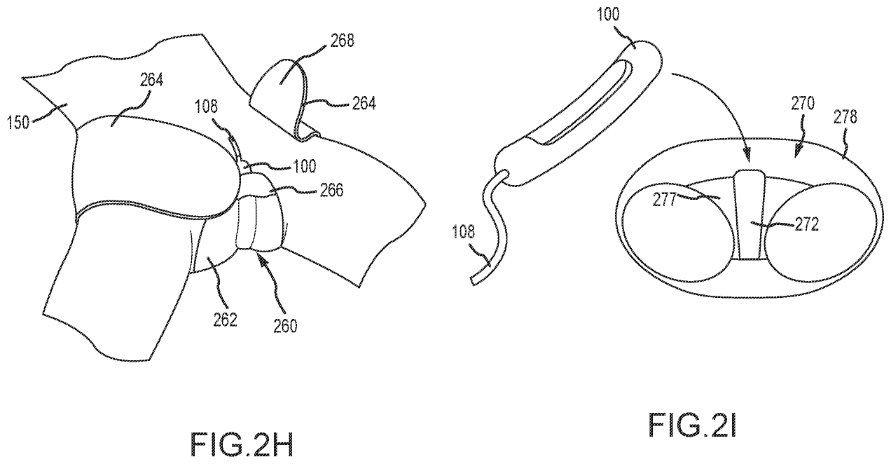

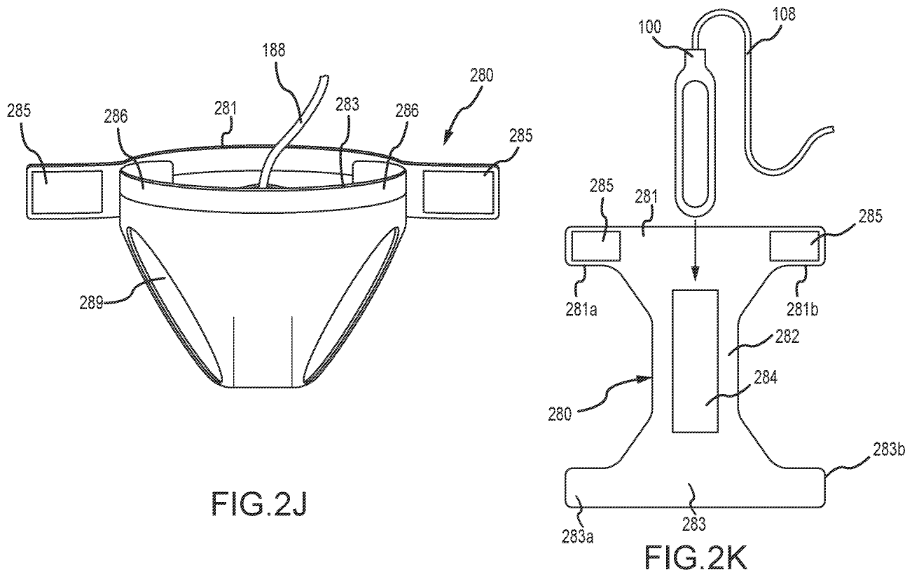

Also disclosed herein are embodiments of means for securing a fluid collection device, such as the fluid collection device 100, to a user with the fluid permeable body positioned proximate (e.g., adjacent) to the urethra of the user through the opening in the fluid collection device. Embodiments of the means for securing the fluid collection device may be adapted or otherwise configured for use with male fluid collection device, female fluid collection device, or both male and female fluid collection devices. In many embodiments, the means for securing the fluid collection device to the user includes at least a garment or undergarment, such as briefs, boxer briefs, a thong, a jock strap, pants, tights, etc. FIGS. 2A-2K provide various examples of garments or undergarments that may be used as means for securing the fluid collection device to the user according to this disclosure. In embodiments having a garment or undergarment that may be used as a means for securing the fluid collection device to the user, the garment or undergarment may be supportive and use a compressive force to press the fluid collection device 100 against the anatomy (e.g., urethra) of the user 150. This compressive force keeps the fluid collection device 100 secure and in a desired position to prevent or inhibit movement of the fluid collection device 100 and leaks that may occur as a result of movement of the fluid collection device 100. With the garment or undergarment pressing the fluid collection device 100 against the anatomy (e.g., urethra) of the user 150, the fluid collection device 100 is held in place even with movement of the user 150.

The material for the garments or undergarments in FIGS. 2A-2K may include any of a variety of materials. In some embodiments, the material for the garment or undergarment may be thin and configured to not absorb any liquid. In some embodiments, the material for the garment or undergarment may include an absorptive material and/or a memory foam materials that conforms to the body of the user 150. In some embodiments, the garment or undergarment may include any of the materials describe above in relation to the fluid permeable body 120, such as a wicking material. The garments or undergarments in FIGS. 2A-2K may be configured for either single-use or multi-use (i.e., washable). One or more embodiments including garments or undergarments as means for securing the fluid collection device to the user may have one or more features that assist or aid in proper placement of the fluid collection device 100.

Turning specifically to FIG. 2A, a fluid collection system may include a fluid collection device 100 and an undergarment 200, according to an embodiment. The undergarment 200 may include a body portion 208 and a pocket flap 202. The body portion 208 defines a pelvic opening 207 positioned on the undergarment 200 to surround or expose the urethra of the user 150 through the pelvic opening 207 when the undergarment 200 is worn by the user 150. The body portion 208 also may include a waist opening and two leg openings. The body portion 208 may include any materials used for undergarments. In some embodiments, the material of the body portion 208 includes at least one of cotton, polyesters, silk, elastane, open fabric weave material, a sponge-type material, or blends thereof.

The pocket flap 202 may be adjustable between an open position (not shown) that exposes the pelvic opening 207 when the undergarment 200 is worn by the user 150 and a closed position (shown in FIG. 2A) that covers the pelvic opening 207. Accordingly, the pocket flap 202 may include a first end secured to the body portion 208 and a second end that is not secured to the body portion 208. For example, the pocket flap 202 may include a first end secured to the body portion 208 between the urethra of the user 150 and the backside of the user 150 and/or the backside of the undergarment 200, and the pocket flap 202 may include a second end on the front side of the undergarment 200 that is not secured to the body portion 208. The second end not secured to the undergarment may be proximate to the waistband of the undergarment 200 on the front side of the undergarment 200, such as shown in FIG. 2A. In some embodiments, other ends and/or sides of the pocket flap 200 may be secured to the body portion 208. For example, an end of the pocket flap 202 may be secured to the front side of the body portion 208, and an end of the pocket flap 202 may not be secured to the body portion 208 on the bottom or back side of the body portion 208. In some embodiments, a side of the pocket flap 202 may be secured to the body portion 208, while an opposing side, the first end, and the second end of the pocket flap 202 are not secured to the body portion 208. In some embodiments, one side of the pocket flap 202 and one of the first end or the second end of the pocket flap 202 are secured to the body portion 208.

The undergarment 200 also includes one or more fasteners 204 configured to selectively and releasably fasten a portion of the pocket flap 202 to the body portion 208. The one or more fasteners 204 and the pocket flap 202 are configured to fasten the pocket flap 202 to the body portion 208 with the fluid collection device 100 positioned between the user 150 and the pocket flap 202 effective to retain the fluid collection device 100 with the fluid permeable body 120 positioned proximate to the urethra of the user 150 through the opening 106 of the fluid collection device 100. In some embodiments, the one or more fasteners 204 may be positioned to releasably fasten the body portion 208 to an end or side of the pocket flap 202 that is not connected or secured to the body portion 208. In the undergarment 200, the one or more fasteners 204 are positioned and configured to releasably fasten the second end of the pocket flap 202 to the body portion 208 proximate to or on a waistband of the undergarment 200. The one or more fasteners 204 may include at least one of hook and loop fasteners, buttons, snaps, a zipper, magnets, or any other fastener.

The pocket flap 202 may include the same or different material than the body portion 208. In some embodiments, the pocket flap 202 is integrally formed with the body portion 208 on one or more sides or ends of the pocket flap 202. In some embodiments, the pocket flap 202 includes a wicking material and in some embodiments the pocket flap 202 includes an absorbent material configured to absorb any fluids leaked or missed by the urine collection device. For example, the pocket flap 202 may include at least one of cotton, polyesters, silk, elastane, open fabric weave material, a sponge-type material, or blends

In use, the pocket flap 202 provides the technical effect of allowing the user 150 or a caregiver to open the pocket flap 202 to insert, remove, or replace a fluid collection device without removing the undergarment 200 from the pelvic region of the user 150. When the pocket flap 202 is closed, the pocket flap 202 provides the technical effect of compressing the fluid collection device 100 against the user 150.

FIGS. 2B and 2C show another embodiment of an undergarment 210 configured to provide the technical effect of retaining the fluid collection device 100 positioned proximate (e.g., adjacent) to the urethra of the user 150. Unless otherwise noted, the undergarment 210 may include any aspect of the undergarment 200 described above. Moreover, the undergarment 200 may include any aspect of the undergarment 210. The undergarment 210 includes a body portion 218 and a pocket flap 212. FIG. 2B shows the pocket flap 212 releasably secured to the body portion 218 in a closed position and FIG. 2C shows the pocket flap 212 in the open position.

The body portion 218 defines a pelvic opening 217 (shown in FIG. 2C) positioned on the undergarment 210 to surround or expose the urethra of the user 150 to the fluid permeable body 120 of the fluid collection device 100 through the pelvic opening 217 when the undergarment 210 is worn by the user 150 and the pocket flap 212 is in the open position. The body portion 218 also defines a waist opening and two legs openings. The pocket flap 212 may be secured, connected, or otherwise attached to the body portion 218 as described above in relation to the garment 200.

The undergarment 210 also includes one or more fasteners. For example, the undergarment 210 includes a first fastener 214 secured to the pocket flap 214 and a second fastener 216 secured to the body portion 218 and positioned to releasably and selectively mate or engage with the first fastener 214 when the pocket flap 212 is in the closed position. The first fastener 214 and the second fastener 216 may include at least one of hook and loop fasteners, buttons, snaps, a zipper, magnets, or any other fasteners.

In use, the pocket flap 212 provides the technical effect of allowing a user or caregiver to open the pocket flap 212 to insert, remove, or replace a fluid collection device without removing the undergarment 210 from the pelvic region of the user 150. When the pocket flap 212 is closed, the pocket flap 212 provides the technical effect of compressing the fluid collection device 100 against the user 150.

FIG. 2D shows another embodiment of an undergarment 220 providing the technical effect of retaining the fluid collection device 100 positioned proximate (e.g., adjacent) to the urethra of the user 150. Unless otherwise noted, the undergarment 220 may include any aspect of the undergarments 200, 210 described above. Moreover, the undergarments 200, 210 may include any aspect of the undergarment 220. The undergarment 220 includes a body portion 228 defining a pelvic opening 227 positioned on the undergarment 220 to expose the urethra of the user 150 to the fluid permeable body 120 of the fluid collection device 100 through the pelvic opening 227 when the undergarment 220 is worn by the user 150. In the garment 220, the pelvic opening 227 may extend all the way from the pelvic region of the user 150 to the waistband of the garment 220. In some embodiments, the garment 220 may include a pelvic opening 227 sized and shaped similarly to the pelvic opening 207, 217. The body portion 218 also defines a waist opening and two legs openings.

The undergarment 220 also includes one or more straps 220 extending or extendable across the pelvic opening 217 and configured to retain the fluid collection device 100 between the user 150 and the one or more straps 222 effective to provide the technical effect of retaining the fluid collection device 100 positioned proximate to the urethra of the user 150. For example, the undergarment 220 includes a first strap 222a extending or extendable across the pelvic opening 227 and positioned to be proximate (e.g., adjacent) to the second end region 127 of the fluid impermeable barrier 102 of the fluid collection device 100, a second strap 222 extending or extendable across the pelvic opening 227 and positioned to be proximate (e.g., adjacent) to the first end region 125 of the fluid impermeable barrier 102 of the fluid collection device 100, and a third strap 222c extending or extendable across the pelvic opening 227 proximate to the waistband of the garment 220. In some embodiments, the conduit 108 may be tucked under the third strap 222c. While the undergarment 220 includes all of the straps 222a, 222b, 222c, other embodiments, may include only one or two of the straps 222a, 222b, 222c. For example, the one or more straps of the undergarment 220 may include only the strap 222a and the strap 222b, the strap 222a and the strap 222c, the strap 222b and the strap 222c, the strap 222a, the strap 222b, or the strap 222c. In some embodiments, the undergarment 220 may include a strap positioned approximately between the strap 222a and the strap 222b shown in FIG. 2D and the straps 222a, 222b may be absent.

The one or more straps 222 may include an elastic and/or a cloth material. In some embodiments, both ends of at least one of the one or more straps 222 may be fixedly secured to the body portion 228 with threading, ultrasonic welding, an adhesive, or another securement feature. In some embodiments, only a single end of at least one of the one or more straps 222 may be fixedly secured to the body portion 228, and a second end of the at least one of the one or more straps 222 may be selectively and releasably fastened or fastenable to the body portion 228. For example, a second end of at least one of the one or more straps 222 may be secured to the body portion 208 with at least one of hook and loop fasteners, buttons, snaps, a zipper, magnets, or any other fastener. In some embodiments, both ends of at least one of the one or more straps 222 may be selectively and releasably fastened or fastenable to the body portion 228. In some embodiments, at least one of the one or more straps 222 may be configured provide the technical effect of allowing a user or caregiver to adjust the undergarment 220 to the specific user. For example, a smaller user may need at least one of the one or more straps 222 tightened further than a larger user. Accordingly the body portion 228 may include an elongated strip of fastening material or multiple buttons, snaps, or magnets that provide the technical effect of allowing the user or caregiver to selectively adjust the tightness of the at least one strap 222 against the user 150.

FIG. 2E shows another embodiment of an undergarment 230 providing the technical effect of retaining the fluid collection device 100 positioned proximate (e.g., adjacent) to the urethra of the user 150. Unless otherwise noted, the undergarment 230 may include any aspect of the undergarments 200, 210, 220 described above. Moreover, the undergarments 200, 210, 220 may include any aspect of the undergarment 230. The undergarment 230 includes a body portion 238 defining a pelvic opening positioned on the undergarment 230 to expose the urethra of the user 150 to the fluid permeable body 120 of the fluid collection device 100 through the pelvic opening when the undergarment 230 is worn by the user 150. The body portion 218 also defines a waist opening and two legs openings.

The undergarment 200 also includes a patch 232 detachably fastened or securable to the body portion 238 over the pelvic opening. The patch 232 may be sized and dimensioned to cover the entire fluid collection device 100 and/or the pelvic opening in the body portion 238 to hold the fluid collection device 100 in place. The patch 232 may include any of the materials described above in relation to the pocket flaps 202, 212. In some embodiments, the patch 232 may include an absorbent material to capture any potential fluid leaks.

The undergarment 200 also may include one or more fasteners 234 configured to selectively and detachably fasten the patch 232 to the body portion 238 with the fluid collection device 100 positioned between the user 150 and the patch 232 effective to provide the technical effect of retaining the fluid collection device 100 positioned proximate to the urethra of the user 150. The one or more fasteners 234 may include at least one of hook and loop fasteners, buttons, snaps, a zipper, magnets, or any other fastener. In some embodiments, the patch 232 and the one or more fasteners 234 provide the technical effect of allowing the compression or tightness of the patch 232 to be adjusted to meet the specific user, as described above in relation to the undergarment 220. The patch 232 also may provide the technical effect of allowing the patch 232 to be entirely removed and replaced with a new patch 232 without removing the undergarment 230 if the patch 232 becomes soiled or otherwise defective.

FIG. 2F shows another embodiment of an undergarment 240 providing the technical effect of retaining the fluid collection device 100 positioned proximate (e.g., adjacent) to the urethra of the user 150. Unless otherwise noted, the undergarment 240 may include any aspect of the undergarments 200, 210, 220, 230 described above. Moreover, the undergarments 200, 210, 220, 230 may include any aspect of the undergarment 240. The undergarment 240 includes a body portion 248 defining a waist opening and two legs openings.

The undergarment 240 also includes one or more pockets 242 secured or securable to an inner surface of the undergarment 240. For example, the undergarment 240 may include a first pocket 242a sized and dimensioned to retain therein at least a portion of the first end region 125 of the fluid impermeable barrier 102, and a second pocket 242b sized and dimensioned to retain therein at least a portion of the second end region 127 of the fluid impermeable barrier 102. The one or more pockets 242 are positioned to provide the technical effect of preventing or inhibiting the fluid collection device 100 from moving up or down and/or side to side when the fluid collection device 100 is positioned at least partially within the one or more pockets 242. For example, the first pocket 242a and the second pocket 242b may be positioned on the inner surface effective to retain the fluid collection device 100 with the fluid permeable body 120 positioned proximate to the urethra of the user through the opening 106 of the fluid collection device 100 when the first end region 125 is positioned in the first pocket 242a and the second end region 127 is positioned in the second pocket 242b.

In some embodiments, at least one of the one or more pockets 242 may be enclosed pockets or straps defining a through hole. The one or more pockets 242 may include an elastic material. In some embodiments, the one or more pockets 242 may be fixedly secured to the inner surface of the undergarment 200 on both sides of the one or more pockets 242 or only one side of the one or more pockets 242. In some embodiments, the one or pockets 242 may be selectively and releasably secured or securable to the inner surface of the undergarment on at least one side of the one or more pockets 242.

FIG. 2G shows another embodiment of an undergarment 250 configured retain the fluid collection device 100 positioned proximate (e.g., adjacent) to the urethra of the user 150. Unless otherwise noted, the undergarment 250 may include any aspect of the undergarments 200, 210, 220, 230, 240 described above. Moreover, the undergarments 200, 210, 220, 230, 240 may include any aspect of the undergarment 250. The undergarment 250 includes a body portion 258 defining a waist opening and two legs openings.

The undergarment 250 also include two alignment pads 254 secured or securable to an inner surface of the undergarment 250. The two alignment pads 254 are spaced to position the fluid collection device 100 between the two alignment pads 254 effective to provide the technical effect of retaining the fluid collection device 100 with the fluid permeable body 120 positioned proximate to the urethra of the user 150 through the opening 106 of the fluid collection device 100. Accordingly, the two alignment pads 254 may be positioned to provide the technical effect of preventing or inhibiting the fluid collection device 100 from moving side-to-side under the undergarment 200.

In some embodiments, the two alignment pads 254 include and absorptive and/or foam material. The material of the two alignment pads may be configured to wick fluid away from the skin of the user. In some embodiments, the two alignment pads may include any of the materials described above in relation to the fluid permeable body 200. The two alignment pads 254 may each include a thickness of less than about 2.5 cm, less than about 1.9 cm, less than about 1.3 cm, at least 0.64 cm, at least about 1.3 cm, at least about 1.9 cm, about 0.64 cm to about 2.5 cm, about 0.64 cm to about 1.3 cm, about 1.3 cm to about 1.9 cm, or about 1.9 cm to about 2.5 cm. The two alignment pads 254 may be fixedly secured to the inner surface of the undergarment 250 with, for example, a threaded seam, ultrasonic welding, an adhesive, or any other securement. In some embodiments, the two alignment pads 254 may be selectively and releasably secured or securable to the inner surface of the undergarment 250 with a fastener, the fastener including at least one of hook and loop fasteners, buttons, snaps, a zipper, magnets, or any other fastener. The two alignment pads 254 also may be incorporated into any of the other undergarments disclosed herein.

FIG. 2H shows another embodiment of an undergarment 260 providing the technical effect of retaining the fluid collection device 100 positioned proximate (e.g., adjacent) to the urethra of the user 150. Unless otherwise noted, the undergarment 260 may include any aspect of the undergarments 200, 210, 220, 230, 240, 250 described above. Moreover, the undergarments 200, 210, 220, 230, 240, 250 may include any aspect of the undergarment 260.

The undergarment 260 may include a T- or Y-shaped undergarment having a body portion 262 and two arms 264 extending from the body portion 262. The body portion 262 is sized to cover at least a portion (e.g., at least most or a majority) of the fluid collection device 100 when the fluid permeably body 120 is positioned proximate to the urethra of the user 150. The body portion 262 also is sized and dimensioned to wrap between legs of the user 150 to a backside of the user 150. In some embodiments, the body portion 262 includes one or more pockets configured to hold the fluid collection device 100 in place on the body portion 262, such as one or more of the pockets 242a, 242b described above.

The two arms 264 each extend from the body portion 262 and are sized to extend from the body portion 262, wrap around a different waist side of the user 150, and fasten to the body portion 262 proximate to the fluid collection device 100 effective to provide the technical effect of retaining the fluid collection device 100 with the fluid permeable body 120 positioned proximate to the urethra of the user 150 through the opening 106 of the fluid collection device 100.

The undergarment 260 also may include one or more fasteners configured to selectively and releasably secure to the two arms 264 to the body portion 262. For example, the undergarment 260 may include a fastener 266 on the outer surface of the body portion 262 and a complementary fastener 268 on the inner surface of each of the two arms 262. The one or more fasteners may include at least one of hook and loop fasteners, buttons, snaps, magnets, or any other fastener. The fasteners 266, 268 may be positioned on the arms 264 and the body portion 262, respectively, to allow the undergarment 260 to be selectively adjusted to the size of individual users when securing the undergarment 260 to the user.

FIG. 2I shows an embodiment of an undergarment 270 providing the technical effect of retaining the fluid collection device 100 positioned proximate (e.g., adjacent) to the urethra of the user 150. Unless otherwise noted, the undergarment 270 may include any aspect of the undergarments 200, 210, 220, 230, 240, 250, 260 described above. Moreover, the undergarments 200, 210, 220, 230, 240, 250, 260 may include any aspect of the undergarment 270. The undergarment 270 includes a body portion 278 defining a waist opening and two legs openings.

The undergarment 270 also includes an inner portion 277, such as a pelvic inner portion, having a recess 272 or depression shaped complementary to at least a portion of the fluid collection device 100. For example, the recess 272 may include an elongated recess curved complementary to a back side (opposite to the opening 106) of the fluid impermeable barrier 102. The recess 272 is positioned and sized such that when at least the portion of the fluid collection device 100 is positioned within the recess 272 and the undergarment 270 is worn by the user 150, the recess 272 of the inner portion 277 provides the technical effect of holding the fluid collection device 100 in place with the fluid permeable body 120 positioned proximate to (e.g., directly against) the urethra of the user through the opening 106 of the fluid collection device 100.

In some embodiments, at least the inner portion 277 having the recess 272 includes a gel or foam material having the recess 272 molded thereon. For example, the inner portion 277 may include a closed cell foam material having the recess 272 molded or otherwise formed thereon. The inner portion 277 may be fixedly secured to the inner surface of the body 278 of the undergarment 270 with, for example, an adhesive, ultrasonic welding, a threaded seam, or combinations thereof. In some embodiments, the inner portion 277 may be selectively and removably secured to the undergarment 270 with, for example, a pocket on the inner surface of the body 278 or hook and loop fastener materials.

FIGS. 2J and 2K show another embodiment of an undergarment 280 configured retain the fluid collection device 100 positioned proximate (e.g., adjacent) to the urethra of the user 150. Unless otherwise noted, the undergarment 280 may include any aspect of the undergarments 200, 210, 220, 230, 240, 250, 260, 270 described above. Moreover, the undergarments 200, 210, 220, 230, 240, 250, 260, 270 may include any aspect of the undergarment 280. The undergarment 280 includes a first end region 281, a second end region 283, an intermediate region 282, and one or more pockets 284 on the intermediate region 282. The undergarment 280 is configured to adjust from an unwrapped or unfolded position, shown in FIG. 2K, to a wrapped or folded position for use, shown in FIG. 2J.

The intermediate region 282 includes one or more pockets positioned at least partially on an inner surface of the intermediate region 282. The undergarment 280 shown in FIG. 2K includes a single elongated pocket 284 sized and dimensioned to receive at least a portion (e.g., most or all) of the fluid collection device 100. In some embodiments, the single elongated pocket 284 may at least partially cover the opening 106 of the fluid collection device 100 when the fluid collection device is at least partially inserted into the single elongated pocket 284. Accordingly, the single elongated pocket 284 may include a wicking material, a mesh material, combinations thereof, or other material configured to allow fluid discharged from the user 150 pass through the material of the pocket 284 to the fluid permeable body 120 of the fluid collection device 100. In some embodiments, single pocket 284 may be sized such that the single pocket 284 does not cover the opening 106 of the fluid collection device 100. In some embodiments, the one or more pockets include multiple pockets, such as the two pockets 242a, 242b described above.

The first end region 281 of the undergarment 280 includes two lateral arms 281a, 281b and a fastener 285 on the inner surface each arm of the two lateral arms 281a, 281b. The fastener 285 may include one or more of hook and loop fasteners, buttons, snaps, an adhesive, magnets, or any other fastener. The second end region 283 is distal to the first end region 281 and including two lateral arms 283a, 283b. Turning to FIG. 2J, the first end region 281 and the second end region 283 are sized and dimensioned such that the fasteners 285 on each arm of the two lateral arms 281a, 281b of the first end region 281 is fastened or fastenable to the outer surface 286 of the lateral arms 283a, 283b and/or the second end region 283 to define leg openings 289 when the intermediate region 282 is positioned proximate to the urethra of the user (not shown).

In use, the undergarment 280 may be positioned in the unwrapped or unfolded position shown in FIG. 2K with the pocket 284 proximate to the urethra of the user 150. The fluid collection device 100 may be inserted into the pocket 284 before or after positioning the unwrapped or unfolded undergarment 280 proximate to the urethra of the user 150. The end regions 281, 283 may then be wrapped around the pelvis and backside of the user, and the fastener 285 on the arms 281a, 281b of the first end region may be secured to the outer surface 286 of the second end region 283 to form the folded or wrapped position of the undergarment 280 on the user 150.

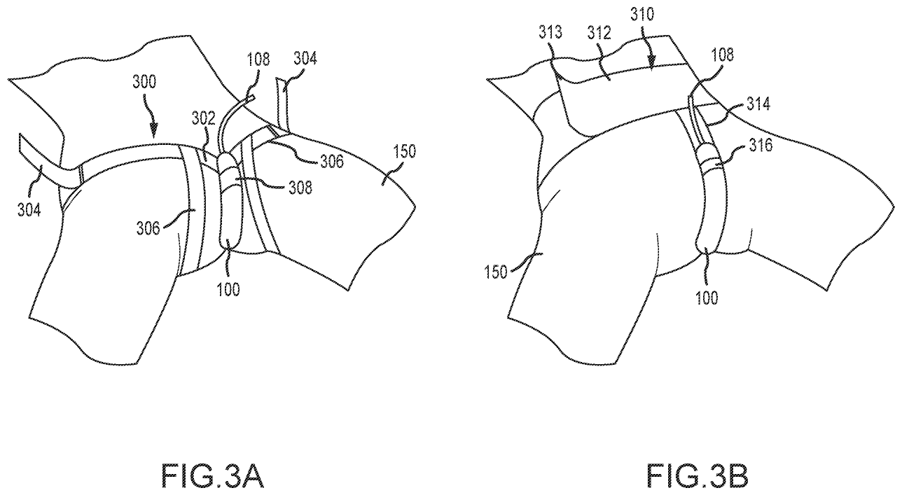

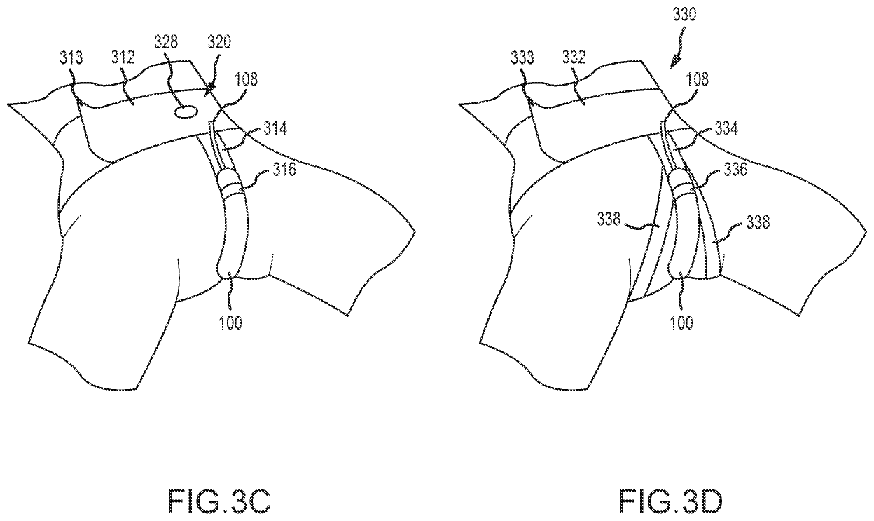

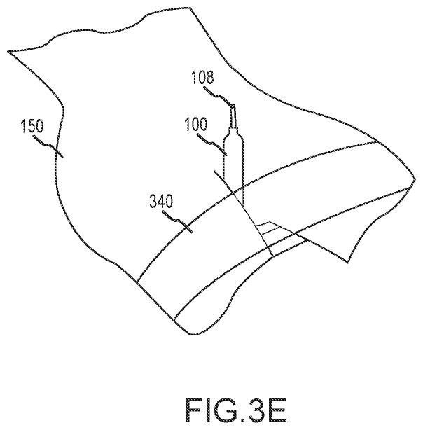

Turning to FIGS. 3A-3E, in some embodiments, the means for securing the fluid collection device to the user may include a securement element having one or more straps. According to the various embodiments disclosed herein, one or more straps may be configured to tie or secure around a fluid collection device and the body of the user to provide the technical effect of securing the fluid collection device in a preferred or desired position on the user. The securement of the fluid collection device in place results in the technical effect of preventing or inhibiting movement of the fluid collection device and leaks that may occur as a result of movement of the fluid collection device while in use. The one or more straps may be more beneficial than other securement elements because the one or more straps have minimal interaction with the skin of the user, thereby decreasing the chance of skin irritation and sores on the user.

FIG. 3A shows an embodiment of a securement element 300 including two leg straps 306 and a central strap 302 connecting or extending between the two leg straps 306. Each leg strap 306 may include a single strap or multiple straps secured or securable together to define a leg opening. Each leg strap 306 is configured to wrap around a leg, an upper thigh, and/or a buttock of the user 150. For example, each leg strap 306 may wrap around the user 150 from the lower abdomen or pelvic region, between the legs, around the back of the thigh or buttock, around the hip of the user 150, and then meet the leg strap 306 again at the lower abdomen or pelvic region.

In some embodiments, each leg strap 306 may be elastic, canvas, rubber, fabric, or any combination thereof. Each leg strap 306 also may be adjustable, providing the technical effect of allowing the user 150 or caregiver to selectively adjust the size of the leg strap 306 depending on the size of the leg(s) of the user 150. In some embodiments, the leg strap 306 may include one or more of a clip, loop, buckle, adhesive hook and loop fasteners, snaps, or any other fastener. The securement element 300 includes a strip 304 of hook and loop fasteners configured to allow the user 150 or caregiver to adjust the size of the leg strap 306 around the leg of the user 150.

The central strap 302 may extend between and/or connect the two leg straps 306. The central strap 302 also includes a loop 308 on the central strap 302 configured to receive and hold at least a portion of the fluid collection device 100 within the loop effective to provide the technical effect of positioning the fluid permeable body 120 proximate to the urethra of the user 150 through the opening 106 of the fluid collection device 100 when the portion of the fluid collection device 100 is held within the loop 308 and the two leg straps 306 are each positioned around a different leg of the user 150. The single connection point of the loop 308 on the central portion 302 to the fluid collection device 100 may result in the technical effect of avoiding discomfort and tendency for the fluid collection device 100 to be pulled away from the anatomy (e.g., urethra) of the user 150. The central portion 302, including the loop 308, may include elastic, canvas, rubber, fabric, or any combination thereof. In some embodiments, the loop 308 stretches to fit around the fluid impermeable barrier 102 to secure a portion of the fluid collection device 100 within the loop 308.

In some embodiments, the central strap 302 may be substantially V- or U-shaped, with the loop 308 positioned at the bottom of the V- or U-shape. The loop 308 may be positioned to wrap around at least a portion of the first end region 125 of the fluid impermeable barrier 102, according to an embodiment. In other embodiments, the loop 308 may be positioned to wrap around other portions of the fluid impermeable barrier 102, such as the second end region 127. Although the securement element 300 shows only a single loop 308, other embodiments of the securement element 300 include a second loop in addition to the loop 308 shown in FIG. 3A. The second loop may be configured to secure to the second end region 127 of the fluid impermeable barrier 102, while the loop 308 may be configured to secure to the first end region 125 of the fluid impermeable barrier 102. In embodiments including a second loop, the second loop may be secured or connected to the central strap 302, or the securement element 300 may include one or more additional straps securing or connecting the second loop to at least one of the two leg straps 306.

FIG. 3B shows an embodiment of a securement element 310 that includes a waist strap 312 and a central strap 314. The waist strap 312 is configured to extend around the waist of the user 150. The waist strap 312 may include any number of materials, such elastic, polyester canvas, rubber, fabric, or any combination thereof. In many embodiments, the waist strap 312 is an adjustable strap, such as an adjustable belt configured to fit users of varying sizes. In some embodiments, the waist strap 312 may include one or more of a clip, loop, buckle, adhesive hook and loop fasteners, snaps, or any other fastener. The waist strap 312 includes a strip 313 of hook and loop fastener material configured to allow the user 150 or caregiver to adjust the size of the waist strap 312 around the waist of the user 150. The waist strap 312 may have a width that is greater than a width of the central strap 314. For example, the waist strap 312 may have a width that is at least about 3.8 cm, at least about 5.1 cm, at least about 6.4 cm, or at least about 7.6 cm, and the central strap may have a width that is less than about 3.8 cm, less than about 3.2 cm, less than about 2.5, less than about 1.9 cm, or less than about 1.3 cm.

The central strap 314 is secured or connected to the waist strap 312 and may, in some embodiments, extend downward generally vertically from the waist strap 312. The central strap 314 also includes a loop 316 secured thereto and configured to receive and hold a portion of the fluid collection device 100 within the loop 316 effective to position the fluid permeable body 120 proximate to the urethra of the user 150 through the opening 106 of the fluid collection device 100 when the portion of the fluid collection device 100 is held within the loop 316 and the waist strap is positioned around the waist of the user 150. In some embodiments, the central strap 314 may be adjustable in length to provide the technical effect of accommodating variations in how the fluid collection device 100 is positioned on the user 150. The central strap 314 may include one or more of a clip, loop, buckle, adhesive hook and loop fasteners, snaps, or any other fastener resulting in the technical effect of allowing the user 150 or caregiver to selectively adjust a length of the central strap 314. The central strap 314 and the loop 316 may include any number of materials, such elastic, polyester canvas, rubber, fabric, or any combination thereof.

The loop 316 may include any aspect of the loop 308 described above. The loop 316 may be positioned to wrap around at least a portion of the first end region 125 of the fluid impermeable barrier 102, according to an embodiment. In other embodiments, the loop 316 may be positioned to around other portions of the fluid impermeable barrier 102, such as the second end region 127. Although the securement element 310 shows only a single loop 316, other embodiments of the securement element 310 include a second loop in addition to the loop 316 shown in FIG. 3B. The second loop may be configured to secure to the second end region 127 of the fluid impermeable barrier 102, while the loop 316 may be configured to secure to the first end region 125 of the fluid impermeable barrier 102. In embodiments including a second loop, the second loop may be secured or connected to the central strap 314, or the securement element 300 may include one or more additional straps securing or connecting the second loop to the waist strap 312.

FIG. 3C shows an embodiment of a securement element 320 that includes the waist strap 312, the central strap 314, and the loop 316. Unless otherwise noted, the securement strap 320 may include any aspect of the securement element 310 described above. In some embodiments, the securement element 320 include an opening 328 defined by the waist strap 312. The opening 328 is positioned on the waist strap 312 to align with a navel of the user 150 resulting in the technical effect of helping aid in placement of the waist strap 312 to make the placement process more repeatable.

FIG. 3D shows an embodiment of a securement element 330 that includes a waist strap 332, a central strap 334, two leg straps 338, and a loop 336 on at least one of the central strap 334 or the leg straps 338. Unless otherwise noted, the securement element 330 may include any aspect of the securement elements 300, 310, or 320. In some embodiments, the securement element 330 may combine any aspect of the waist belts, the central straps, and/or the leg straps of other securement elements 300, 310, or 320 described herein. By combining these aspects of other securement elements 300, 310, or 320, the variability when positioning the fluid collection device 100 and the securement element 330 may be decreased.

The waist strap 332 is configured to extend around a waist of the user 150 and may include any aspect of the waist strap 312. In some embodiments, the waist strap 332 may include one or more of a clip, loop, buckle, adhesive hook and loop fasteners, snaps, or any other fastener. The securement element 330 includes the strip 333 of hook and loop fasteners configured to allow the user 150 or caregiver to adjust the size of the waist strap 332 around the waist of the user 150.

The central strap 334 may extend downward vertically from the waist strap 332. The two leg straps 338 may be configured to wrap around the user 150 from the lower abdomen or pelvic region, between the legs, and around the back of the thigh or buttock of the user 150. In the securement element 330, each leg strap 338 may be secured to the central strap 334 and extends to wrap around the user 150 from the lower abdomen or pelvic region, between the legs, and around the back of the thigh or buttock of the user 150 where the leg strap 338 is secured to a back region of the waist strap 332. In some embodiments, each leg strap 338 is secured to the central strap 334 and extends to wrap around the user 150 from the lower abdomen or pelvic region, between the legs, around the hip of the user 150, and then meet the leg strap 338 or the central strap 334 again at the lower abdomen or pelvic region.

The loop 336 may include any aspect of the loops 308, 316 and may be configured to receive and hold a portion of the fluid collection device 100 within the loop 336 effective to result in the technical effect of positioning the fluid permeable barrier 102 proximate to the urethra of the user 150 through the opening 106 of the fluid collection device 100 when the portion of the fluid collection device 100 is held within the loop 336, the waist strap is positioned around the waist of the user, and the two leg straps 338 are each positioned around a different leg of the user 150.

FIG. 3E shows an embodiment of a securement element including an adjustable strap 340 configured to fit around legs of the user 150 effective to prevent the user 150 from separating the legs. The adjustable strap 150 may prevent or otherwise inhibit the user 150 from unintentionally separating her legs during use. By holding the legs of the user 150 closed, the adjustable strap 150 provides the technical effect of positioning the anatomy (e.g., urethra) of the user 150 to more effectively hold the fluid collection device 100 in place. The adjustable strap 340 can be made of different materials such as a self-adherent wrap, cloth material, elastic, foam, canvas, polyester, or combinations thereof. The adjustable strap 340 also may include one or more of a clip, loop, buckle, adhesive, hook and loop fasteners, snaps, or any other fastener configured to allow the user 150 or caregiver to adjust the size of the adjustable strap 340 around the legs of the user 150.

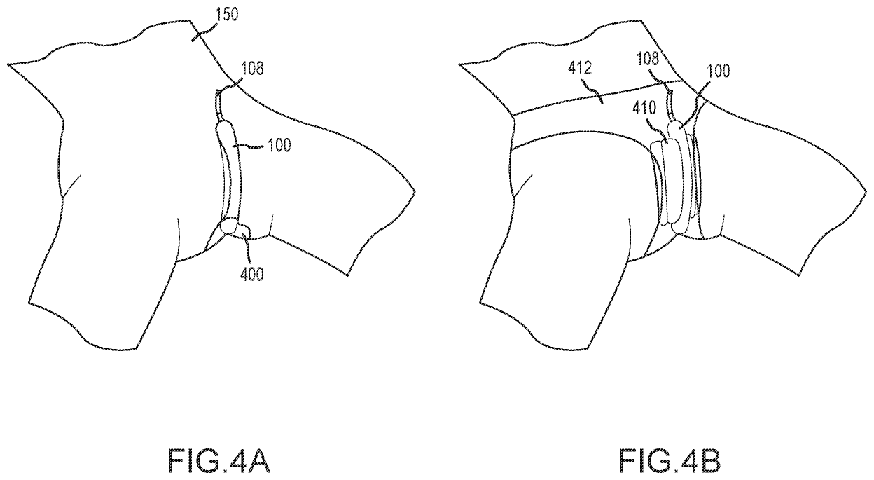

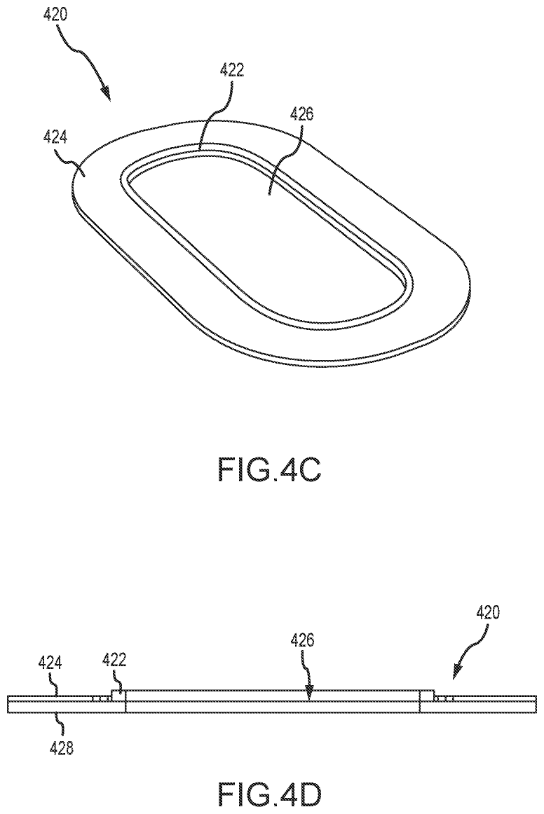

Turning to FIGS. 4A-4D, in some embodiments, the means for securing the fluid collection device to the user may include a securement element having an adhesive element configured to secure the fluid collection device in a desired position on the user. The securement of the fluid collection device in the desired position using the adhesive prevents or inhibits movement of the fluid collection device and leaks that may occur as a result of movement of the fluid collection device while in use. The adhesive can be placed in different areas on the body or clothes of the user, such as on the stomach, along the groin, at least partially on the buttocks, attached to the underwear, and/or on a device secured to the fluid collection device. For example, the adhesive may include a patch configured to adhere to the conduit of the fluid collection device and the stomach or abdomen of the user. In another embodiment, the adhesive may include a patch configured to adhere to along the groin of the user and the fluid impermeable barrier. The patch may be formed integrally with the conduit or the fluid impermeable barrier, or may be a secondary accessory adhered to the conduit or the fluid impermeable barrier.

FIG. 4A shows an embodiment of an adhesive element that includes an adhesive patch 400 secured or securable to the second end region 127 of the fluid impermeable barrier 102 and configured to adhere to at least one of a gluteal cleft or buttock of the user 150. The adhesive patch 400 may be formed integrally with the fluid impermeable barrier 102, or may be a secondary accessory adhered to the fluid impermeable barrier 102. The adhesive patch 400 also may be sized and dimensioned to position over at least a portion of the second end region 127 of the fluid impermeable barrier 102 and at least one of a gluteal cleft or buttocks of the user 150. In some embodiments, the adhesive patch 400 includes a pocket or elastic strip sized to receive and secure at least a portion of the second end region 127 of the fluid impermeable barrier 102 therein. In some embodiments, the second end region 127 may friction fit within the pocket on the adhesive patch or at least one of the pocket or the second end region 127 may include an adhesive that secures the distal end region 127 to the adhesive patch.

FIG. 4B shows an embodiment of an adhesive element that includes at least one adhesive patch 410 attached or attachable to an undergarment 412 of the user 150. For example, the at least one adhesive patch 410 may be formed or configured as at least one wing. FIG. 4B shows an embodiment having two wings formed from the at least one adhesive patch 410. The two wings may be formed from a single adhesive patch or two separate adhesive patches. In some embodiments, the at least one adhesive patch 410 includes a first region or side secured or securable longitudinally to the fluid impermeable barrier 102 distal to the opening 106 of the fluid collection device 100. The at least one adhesive patch 410 may be fixedly secured or formed integrally with the fluid impermeable barrier 102, or the at least one adhesive patch 410 may be secured or securable to the fluid impermeable barrier 102 with an adhesive on the first region of the at least one adhesive patch 410. In some embodiments, the at least one adhesive patch 410 may be fixedly secured to the inner surface of the undergarment 412, or the at least one adhesive patch 410 may be secured or securable to the inner surface of the undergarment 412 with an adhesive on a second region of the at least one patch 410. In some embodiments, the at least one patch may include a third region having an adhesive configured to temporarily adhere to at least one thigh of the user 150.

FIGS. 4C to 4D show an embodiment of an adhesive element configured as a gasket 420 secured or securable to the fluid impermeable barrier 102 of the fluid collection device 100. The gasket 420 may be configured to create a seal that prevents leaks from the fluid collection device 100, while also securing the fluid collection device 100 in place against the anatomy (e.g., urethra) of the user 150. The gasket 420 provides the technical effect of preventing a decrease an effectiveness of an adhesive if the adhesive gets exposed to watery solutions such as sweat or urine. The gasket 420 includes an outer surface 428, an inner surface 424 including an adhesive, and an elongated opening 426. The adhesive may include any adhesive known in the art, such as a hydrogel. The outer surface 428 may include a thin, flexible backing material, such as a thermoset or a thermoplastic. In some embodiments, the gasket 420 includes a foam material and/or a series of multiple blades and ridges configured to create a gasket effect around the fluid collection device 100.

The elongated opening 426 may be sized complementary to the opening 106 of the fluid impermeable barrier 102 or complementary to an outer periphery of the fluid impermeable barrier 102. For example, if the fluid collection device 100 is positioned proximate to the outer surface 428 of the gasket 420, the elongated opening 426 of the gasket 420 may be aligned with or adjacent to the edge of the opening 106, or the elongated opening 426 of the gasket 420 may be aligned with or adjacent to the outer periphery of the fluid impermeable barrier 102. In some embodiments, the elongated opening 426 is defined at least partially by a seal 422, such as a foam seal or in-mold gasket/isolator.

The gasket 420 may be fixedly secured to the fluid impermeable barrier 102. For example, the gasket 420 may be molded or welded (e.g., ultrasonic welding or radio frequency welding) to the fluid impermeable barrier 102. In some embodiments, the gasket 420 may be molded to the fluid collection device 100 with the inner surface 424 including the adhesive and the opening 106 of the fluid collection device 100 oriented in a common direction to face the user 150 in use. In some embodiments, the gasket 420 may be secured to the fluid impermeable barrier 102 with an adhesive or mechanical attachment. For example the seal 422 may include an adhesive or the inner surface 424 having the adhesive may adhere to both the user 150 and the fluid impermeable barrier 102 to secure the fluid collection device 100 to the user 150.

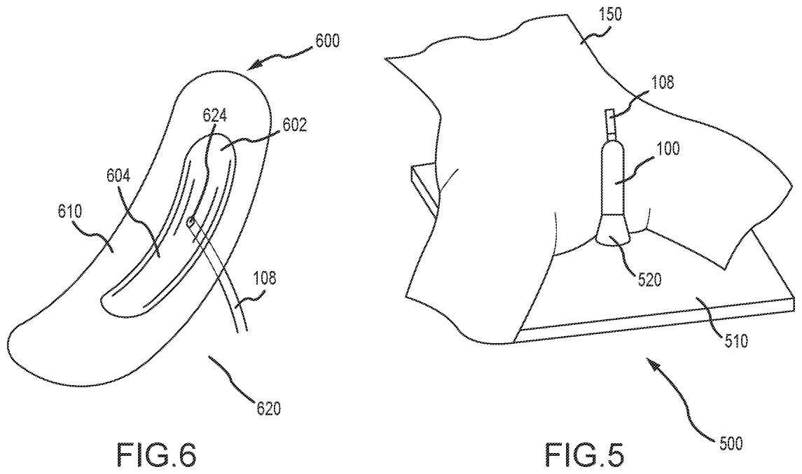

Turning to FIG. 5, in some embodiments, the means for securing the fluid collection device to the user may include a securement element having a stationary surface configured to secure to the fluid collection device to prevent or inhibit movement of the fluid collection device. For example, the securement element 500 may include a base 510 and a receiver 520 secured or securable to the base 510. The base 510 may include a platform having a generally planar or flat surface. In some embodiments, the base 510 may include a material such as foam or gel configured to mold or conform to a portion of the body of the user 150, such as the buttocks and the thighs of the user 150. In some embodiments, the base 510 may include a width of at least about 15 cm and a length of at least 15 cm. For example, the base 510 may include a width of at least about 23 cm, at least about 30 cm, at least about 38 cm, at least about 45 cm, at least about 53 cm, at least about 61 cm, about 15 cm to about 61 cm, about 15 cm to about 30 cm, about 30 cm to about 45 cm, or about 45 cm to about 61 cm. The base 510 may include a length of at least about 23 cm, at least about 30 cm, at least about 38 cm, at least about 45 cm, at least about 53 cm, at least about 61 cm, about 15 cm to about 61 cm, about 15 cm to about 30 cm, about 30 cm to about 45 cm, or about 45 cm to about 61 cm.

The receiver 520 may be sized and dimensioned to receive at least a portion of the second end region 127 of the fluid impermeable barrier 102 and hold the fluid collection device 100 in an angled position relative to the base 510. For example, the receiver 520 may include a recess sized and dimensioned to allow at least a portion of the second end region 127 to be press fit into the recess to removably secure the fluid collection device 100 to the receiver 520. When secured to the receiver 520, the fluid collection device 100 may be positioned to provide the technical effect of allowing the user 150 to lay or sit on the base 510 and have the fluid collection device 100 positioned against the anatomy (e.g., urethra) of the user 150. For example, the securement element 500 may be positioned on the seat of a wheelchair and allow the user 150 to sit on the wheelchair with the fluid collection device 100 positioned against the anatomy (e.g., urethra) of the user 150. In some embodiments, the receiver 520 may be configured and positioned on the base 510 to hold the fluid collection device 100 substantially perpendicular to the surface of the base 510. The receiver 520 may be fixedly secured to the base 510 or removably secured to the base 510 with, for example, a mechanical fastener, a suction cup, an adhesive, or combinations thereof.

Turning to FIG. 6, in some embodiments, the means for securing the fluid collection device to the user may include a securement element having at least a portion of the fluid collection device that is moldable to the user 150 resulting in the technical effect of improved securement of the fluid collection device to the user 150. For example, a fluid collection device 600 may include a heat moldable fluid impermeable barrier 602 defining a recessed chamber 604. In some embodiments, the fluid impermeable barrier 602 also includes an outer flange 610 extending at least partially (e.g., entirely) around the recessed chamber 604. The outer flange 610, however, may include a material different than the fluid impermeable barrier 602. The recessed chamber includes an aperture 624 size to receive at least a portion of the conduit 108 therein effective provide fluid communication between the chamber 604 and the conduit 108.

The fluid impermeable barrier 602 may include a heat-moldable material configured to mold to a shape of the user 150 with the recessed chamber positioned proximate to a urethra of the user 150 when the fluid impermeable barrier is heated to a predetermined temperature. For example, fluid impermeable barrier 602 may be molded to the user 150 such that the opening of the chamber 604 surround the urethra of the user 150 and the chamber 604 is adjacent or proximate to the user 150. The material of the heat moldable fluid impermeable barrier may include any heat moldable material suitable for use next to the skin of the user. In some embodiments, the heat moldable fluid impermeable barrier 602 may include a polyester thermoplastic or other plastic or rubber materials. The fluid impermeable barrier 102 may be configured to be moldable at a predetermined temperature, such as greater than about 90° F., greater than about 100° F., greater than about 125° F., greater than about 150° F., greater than about 175° F., greater than about 200° F., greater than about 225° F., about 90° F. to about 225° F., about 90° F. to about 125° F., about 100° F. to about 125° F., about 125° F. to about 150° F., about 150° F. to about 175° F., about 200° F. to about 225° F., less than about 225° F., less than about 200° F., less than about 175° F., less than about 150° F., less than about 125° F., or less than about 100° F.

Having a heat moldable fluid impermeable barrier 602 provides the technical effect of allowing a user 150 or caregiver to better form the fluid collection device 600 to the individual and unique shapes or contours of the user 150 in order to better secure the fluid collection device 600 to the user 150. In some embodiments, the heat moldable material of the fluid impermeable barrier 102 may be moldable or conformable to the user 150 after inserting the fluid impermeable barrier in water heater to a predetermined temperature for a predetermined amount of time. In some embodiments, the heat moldable material may be moldable or conformable to the body of the user 150 with the body heat of the user 150. A heat moldable fluid impermeable barrier 602 also allows for passive attachment of the fluid collection device 600 to the user 150, i.e., adhesive and/or straps may be absent from the fluid collection device 600. In some embodiments, however, straps or an adhesive may be used in combination with the fluid collection device 600. For example, the outer flange 610 may include an adhesive surface configured to secure to the user 150.

In some embodiments, a fluid permeable body is absent from the fluid collection device 600. In other embodiments, the fluid collection device 600 includes a fluid permeable body positioned in the recessed chamber 604 to wick or direct discharged fluid (e.g., urine) to the aperture 624 and the conduit 108 for removal from the chamber 604. The fluid permeable body may include any aspects of the fluid permeable body 120 described above. The fluid impermeable barrier 102 of the fluid collection device 100 also may include any of the heat moldable materials described herein.

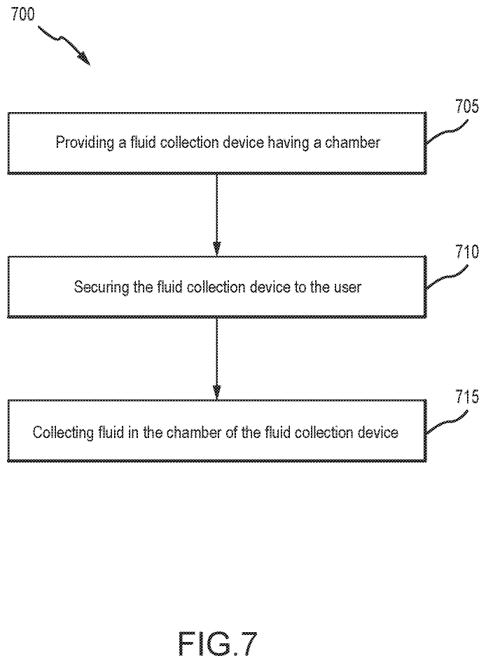

FIG. 7 is a flow diagram of a method 700 for collecting fluids. The method 700 includes an act 705 of providing a fluid collection device having a chamber. The method also includes an act 710 of securing the fluid collection device to the user. The method also may include an act 715 of collecting fluid in the chamber of the fluid collection device. In some embodiments, the method 700 may include an act of applying suction effective to suction the fluids from the chamber via a conduit disposed therein. Acts 705, 710, and 715 of the method 700 are for illustrative purposes. For example, the act 705, 710, and 715 of the method 700 may be performed in different orders, split into multiple acts, modified, supplemented, or combined. Any of the acts 705, 710, and 715 may include using any of the fluid collection devices or systems disclosed herein.

The act 705 may include providing any of the fluid collection devices disclosed herein, such as the fluid collection device 100. For example, the act 705 may include providing a fluid collection device including a fluid impermeable barrier and a fluid permeable body. The fluid impermeable barrier of the fluid collection device provided in the act 705 may at least partially define a chamber, an opening extending longitudinally along the fluid impermeable barrier and configured to be positioned adjacent to a urethra of the user, and an aperture configured to receive a conduit therethrough. The fluid permeable body of the fluid collection device provided in the act 705 may be positioned at least partially within the chamber to extend across at least a portion of the opening and be configured to wick fluid away from the opening.

The act 710 may include securing the fluid collection device to the user with the fluid permeable body positioned proximate to a urethra of either a female or male user user through the opening with one or more means for securing the fluid collection device to the user disclosed herein. In some embodiments, the act 710 may include securing the fluid collection device to the user with the fluid permeable body positioned proximate to a urethra of the user through the opening with an undergarment, such as the undergarment 200, 210, 220, 230, 240, 250, 260, 270, or 280. In some embodiments, the act 710 may include securing the fluid collection device to the user with the fluid permeable body positioned proximate to a urethra of the user through the opening with one or more straps, such as the one or more straps of the securement elements 300, 310, 320, 330, 340. In some embodiments, the act 710 may include securing the fluid collection device to the user with the fluid permeable body positioned proximate to a urethra of the user through the opening with one or more adhesive elements, such as the adhesive elements 400, 410, 420. In some embodiments, the method 700 further comprises an act of positioning a base under at least a pelvic region of the user, and the act 710 may include inserting an end region of the fluid impermeable barrier into a receiver positioned on the base, such as the securement element 500.

In some embodiments, the act 705 may include providing the fluid collection device 600. The act 710 of securing the fluid collection device to the user may include heating a fluid impermeable barrier to a predetermined temperature and molding the fluid impermeable barrier to the user with the recessed chamber positioned proximate to a urethra of the user.

FIG. 8 is a block diagram of a system 10 for fluid collection, according to an embodiment. The system 10 includes a fluid collection device 12, a fluid storage container 14, and a portable vacuum source 16. The fluid collection device 12 may include any of the fluid collection devices described herein, such as the fluid collection device 100 or the fluid collection device 600. The fluid collection device 12, the fluid storage container 14, and the portable vacuum source 16 may be fluidly coupled to each other via one or more conduits 17. The conduit 17 may include any of the conduits described herein, such as the conduit 108. The fluid collection device 12 may be operably coupled to one or more of the fluid storage container 14 or the portable vacuum source via the conduit 17. Fluid (e.g., urine or other bodily fluids) collected in the fluid collection device 12 may be removed from the fluid collection device 12 via the conduit 17, which protrudes into an interior region of the fluid collection device 12. For example, a first open end of the conduit 17 may extend into the fluid collection device 12 to a reservoir therein. The second open end of the conduit 17 may extend into the fluid storage container 14 or the portable vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the portable vacuum source 16. Accordingly, the portable vacuum source 16 may apply suction to the fluid collection device 12 via the fluid storage container 14. The suction force may be applied directly via the fluid storage container 14. For example, the second open end of the conduit 17 may be disposed within the portable vacuum source 16. An additional conduit 17 may extend from the portable vacuum source 16 to a point outside of the fluid collection device 12, such as to the fluid storage container 14. In such examples, the portable vacuum source 16 may be disposed between the fluid collection device 12 and the fluid storage container 14.

The fluid collection device 12 may be shaped and sized to be positioned adjacent or proximate to a female urethra. The fluid collection member of the fluid collection device 12 may include a fluid impermeable barrier at least partially defining a chamber (e.g., interior region of the fluid collection device member) of the fluid collection device 12. As described in more detail above, the fluid collection device 12 may include a softer, thinner fluid impermeable barrier than conventional fluid collection devices. The fluid impermeable barrier also defines an opening extending therethrough from the external environment. The opening may be positioned on the fluid collection member to be aligned adjacent or proximate to a female urethra. The fluid collection member of the fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e.g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the portable vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection member of the fluid collection device 12. In some embodiments, the portable vacuum source 16 may be powered by one or more of a power cord (e.g., connected to a power socket), one or more batteries, or even manual power (e.g., a hand operated vacuum pump). In examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source 16. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

1. A fluid collection assembly, comprising:
a fluid collection device including a fluid impermeable barrier defining a chamber, an opening having fluid communication with the chamber and configured to be placed proximate to a urethra of a user, and an aperture configured to receive a conduit therethrough to provide fluid communication between the chamber and the conduit; and
means for securing the fluid collection device to the user with the opening positioned proximate to the urethra of the user.

2. The fluid collection assembly of claim 1, wherein the means for securing the fluid collection device to the user includes at least an undergarment.

3. The fluid collection assembly of claim 2, wherein the undergarment includes:
a body portion defining a pelvic opening positioned on the undergarment to expose the urethra of the user through the pelvic opening when the undergarment is worn by the user;
a pocket flap having a first end secured to the body portion and a second end, the pocket flap being adjustable from an open position that exposes the pelvic opening and a closed position that covers the pelvic opening; and
one or more fasteners configured to releasably fasten the second end of the pocket flap to the body portion in the closed position with the fluid collection device positioned between the user and the pocket flap effective to retain the fluid collection device with the opening of the fluid impermeable barrier positioned proximate to the urethra of the user.

4. The fluid collection assembly of claim 2, wherein the undergarment includes:
a body portion defining a pelvic opening positioned on the undergarment to expose the urethra of the user through the pelvic opening when the undergarment is worn by the user; and
one or more straps extending across the pelvic opening and configured to retain the fluid collection device between the user and the one or more straps effective to retain the fluid collection device with the opening of the fluid impermeable barrier positioned proximate to the urethra of the user.

5. The fluid collection assembly of claim 2, wherein the undergarment includes:
a body portion defining a pelvic opening positioned on the undergarment to expose the urethra of the user through the pelvic opening when the undergarment is worn by the user;
a patch detachably fastened to the body portion over the pelvic opening; and
one or more fasteners configured to detachably fasten the patch to the body portion with the fluid collection device positioned between the user and the patch effective to retain the fluid collection device with the opening of the fluid impermeable barrier positioned proximate to the urethra of the user.

6. The fluid collection assembly of claim 2, wherein:
the fluid collection device includes a first end region defining the aperture and a second end region distal to the first end region; and
the undergarment includes an inner surface positioned to interface the user, a first pocket sized and dimensioned to retain the first end region therein, and a second pocket sized and dimensioned to retain the second end region therein, the first pocket and the second pocket positioned on the inner surface effective to retain the fluid collection device with the opening of the fluid impermeable barrier positioned proximate to the urethra of the user when the first end region is positioned in the first pocket and the second end region is positioned in the second pocket.

7. The fluid collection assembly of claim 2, wherein the means for securing the fluid collection device to the user includes the undergarment and two alignment pads secured to an inner surface of the undergarment, the two alignment pads being spaced to position the fluid collection device between the two alignment pads effective to retain the fluid collection device with the opening of the fluid impermeable barrier positioned proximate to the urethra of the user.

8. The fluid collection assembly of claim 7, wherein the two alignment pads include and absorptive foam material.

9. The fluid collection assembly of claim 2, wherein the undergarment includes:
a body portion sized to cover at least a portion of the fluid collection device when the fluid permeably layer is positioned proximate to the urethra and wrap between legs of the user to a backside of the user; and
two arms each extending from the body portion and including a fastener, each of the two arms sized to extend from the body portion, wrap around a different waist side of the user, and fasten to the body portion proximate to the fluid collection device effective to retain the fluid collection device with the opening of the fluid impermeable barrier positioned proximate to the urethra of the user.

10. The fluid collection assembly of claim 2, wherein the undergarment includes an inner portion having a recess shaped complementary to at least a portion of the fluid collection device such that when at least the portion of the fluid collection device is positioned within the recess and the undergarment is worn by the user, the recess holds the fluid collection device with the opening of the fluid impermeable barrier positioned proximate to the urethra of the user.

11. The fluid collection assembly of claim 10, wherein the undergarment includes a gel or foam portion having the recess molded therein.

12. (canceled)

13. (canceled)

14. The fluid collection assembly of claim 1, wherein the means for securing the fluid collection device to the user includes one or more straps.

15. The fluid collection assembly of claim 14, wherein the one or more straps includes two leg straps, a central strap connecting the two leg straps, and a loop on the central strap configured to receive and hold a portion of the fluid collection device within the loop effective to position the opening of the fluid impermeable barrier proximate to the urethra of the user when the portion of the fluid collection device is held within the loop and the two leg straps are each positioned around a different leg of the user.

16. The fluid collection assembly of claim 15, wherein the two leg straps are selectively adjustable.

17. The fluid collection assembly of claim 14, wherein the one or more straps include a waist strap configured to extend around a waist of the user, a central strap extending from the waist strap, and a loop on the central strap configured to receive and hold a portion of the fluid collection device within the loop effective to position the opening of the fluid impermeable barrier proximate to the urethra of the user when the portion of the fluid collection device is held within the loop and the waist strap is positioned around the waist of the user.

18. The fluid collection assembly of claim 17, wherein the waist strap and the central strap are selectively adjustable.

19. (canceled)

20. The fluid collection assembly of claim 14, wherein:
the one or more straps include a waist strap configured to extend around a waist of the user, a central strap extending from the waist strap, two leg straps extending from the central strap to the waist strap, and a loop on at least one of the central strap or the two leg straps, the loop being configured to receive and hold a portion of the fluid collection device within the loop effective to position the opening of the fluid impermeable barrier proximate to the urethra of the user when the portion of the fluid collection device is held within the loop, the waist strap is positioned around the waist of the user, and the two leg straps are each positioned around a different leg of the user.

21. The fluid collection assembly of claim 20, further comprising one or more straps connected to the two leg straps and secured or securable to the fluid collection device.

22. The fluid collection assembly of claim 14, wherein the one or more straps include an adjustable strap configured to fit around legs of the user effective to prevent the user from separating the legs.

23. The fluid collection assembly of claim 1, wherein the means for securing the fluid collection device to the user include one or more adhesive elements.

24. The fluid collection assembly of claim 23, wherein:
the fluid impermeable barrier includes a first end region defining the aperture and a second end region distal to the first end region; and
the one or more adhesive elements include an adhesive patch secured or securable to the second end region of the fluid impermeable barrier and configured to adhere to a gluteal cleft or buttock of the user.

25. The fluid collection assembly of claim 23, wherein the one or more adhesive elements include an adhesive patch having:
a first region secured or securable longitudinally to the fluid impermeable barrier distal to the opening; and
a second region including an adhesive configured to adhere to an inner surface of an undergarment.

26. The fluid collection assembly of claim 23, wherein the one or more adhesive members includes a gasket secured or securable to the fluid impermeable barrier, the gasket including an outer surface, an inner surface including an adhesive, and a seal at least partially defining an elongated opening sized complementary to the opening of the fluid impermeable barrier or an outer periphery of the fluid impermeable barrier.

27. The fluid collection assembly of claim 26, wherein the gasket is molded to the fluid impermeable barrier of the fluid collection device with the inner surface including the adhesive and the opening of the fluid collection device oriented in a common direction.

28. The fluid collection assembly of claim 26, wherein the gasket is positioned adjacent to the fluid collection device with the inner surface including the adhesive and the opening of the fluid collection device oriented in a common direction.

29. The fluid collection assembly of claim 1, wherein:
the fluid impermeable barrier includes a first end region defining the aperture and a second end region distal to the first end region; and
the means for securing the fluid collection device to the user includes:
a base; and
a receiver secured or securable to the base, the receiver sized and dimensioned to receive at least a portion of the second end region of the fluid impermeable barrier and hold the fluid collection device in an angled position relative to the base.

30. (canceled)

31. (canceled)

32. The fluid collection assembly of claim 29, wherein the base includes a foam or gel material configured to conform to a portion of the user.

33. (canceled)

34. (canceled)

35. (canceled)

36. A method of collecting fluid from a user, the method comprising:
providing a fluid collection device including a fluid impermeable barrier defining a chamber, an opening having fluid communication with the chamber and configured to be placed proximate to a urethra of a user, and an aperture configured to receive a conduit therethrough to provide fluid communication between the chamber and the conduit;
securing the fluid collection device to the user with the opening positioned proximate to the urethra of the user; and
collecting fluid in the chamber of the fluid collection device,
wherein securing the fluid collection device to the user includes at least one of:
securing the fluid collection device to the user with the opening positioned proximate to the urethra of the user with an undergarment having a pelvic opening,
securing the fluid collection device to the user with the opening positioned proximate to the urethra of the user with one or more straps, or
securing the fluid collection device to the user with the opening positioned proximate to the urethra of the user with one or more adhesive elements, the one or more adhesive elements including a gasket secured or securable to the fluid impermeable barrier, the gasket including an outer surface, an inner surface including an adhesive, and a seal at least partially defining an elongated opening sized complementary to the opening of the fluid impermeable barrier or an outer periphery of the fluid impermeable barrier.

37. (canceled)

38. (canceled)

39. (canceled)

40. The method of claim 36, further comprising positioning a base under at least a pelvic region of the user, wherein securing the fluid collection device to the user with the opening positioned proximate to the urethra of the user includes inserting an end region of the fluid impermeable barrier into a receiver positioned on the base.

41. (canceled)

42. The method of claim 36, wherein securing the fluid collection device to the user with the opening positioned proximate to the urethra of the user includes:
heating a fluid impermeable barrier to a predetermined temperature, the fluid impermeable barrier defining a recessed chamber and having a flanged surrounding the recessed chamber; and
molding the fluid impermeable barrier to the user with the recessed chamber and the opening positioned proximate to the urethra of the user.

43. The method of claim 36, wherein:
providing a fluid collection device including a fluid impermeable barrier defining a chamber, an opening having fluid communication with the chamber and configured to be placed proximate to a urethra of a user, and an aperture configured to receive a conduit therethrough to provide fluid communication between the chamber and the conduit includes:
providing the fluid collection device including the fluid impermeable barrier defining the chamber, the opening having fluid communication with the chamber and configured to be placed proximate to the urethra of a female user, and the aperture configured to receive the conduit therethrough to provide fluid communication between the chamber and the conduit; and
securing the fluid collection device to the user with the opening positioned proximate to the urethra of the user includes:
securing the fluid collection device to the user with the opening positioned proximate to the urethra of the female user.

44. The method of claim 36, wherein:
providing a fluid collection device including a fluid impermeable barrier defining a chamber, an opening having fluid communication with the chamber and configured to be placed proximate to a urethra of a user, and an aperture configured to receive a conduit therethrough to provide fluid communication between the chamber and the conduit includes:
providing the fluid collection device including the fluid impermeable barrier defining the chamber, the opening having fluid communication with the chamber and configured to be placed proximate to the urethra of a male user, and the aperture configured to receive the conduit therethrough to provide fluid communication between the chamber and the conduit; and
securing the fluid collection device to the user with the opening positioned proximate to the urethra of the user includes:
securing the fluid collection device to the user with the opening positioned proximate to the urethra of the male user.
